(19) United States
(12) Patent Application Publication
(10) Pub. No.: US 2003/0026845 A1
(43) Pub. Date: Feb. 6, 2003

(54) Process for preparing protein isolate from milk, whey, colostrum, and the like

(76) Inventors: Lisa R. Etzel, Ames, IA (US); Ronald E. Strohbehn, Nevada, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 09/883,648

(22) Filed: Jun. 18, 2001

(51) Int. Cl. A61K 35/20

(52) U.S. Cl. 424/535

(57) ABSTRACT

According to the invention, Applicants have identified a clarification process which is simple and quick and provides a protein isolate of greater than 90% whey protein which may then optionally be further purified to select out individual proteins. The process involves a number of steps, the order of which is critical, and novel lipid removal techniques to achieving the highly pure and clarified protein isolate of the invention.

### BACKGROUND OF THE INVENTION

The proteins present in milk, colostrum, whey and other compositions produced from lactating animals are of value for their nutritional and functional properties and are often used as ingredients in processed and prepared foods as well as nutritional supplements and even pharmaceutical formulations. These proteins are generally categorized into two classes. The first class is a heterogenous mixture called casein and represents approximately 80% of the proteins found in milk compositions. The second class is a heterogenous mixture called whey proteins comprising the remaining 20% of the proteins in milk.

These proteins are currently separated from milk using a variety of chemical and physical processing techniques. The isolation of these valuable proteins from milk compositions has proven complicated and difficult for researchers. The proteins in order to retain their activity must not be damaged or denatured during the purification process and thus harsh treatments such as heat or lengthy exposure to strong acid must be avoided. Thus isolation of proteins has centered around the concepts of filtration or ion exchange techniques.

When a solution and water are separated by a semi-permeable membrane, the water will move into the solution to equilibrate the system. This is known as “osmotic pressure”. If a mechanical force is applied to exceed the osmotic pressure (up to 700 psi), the water is forced to move down the concentration gradient i.e. from low to high concentration. “Permeate” designates the liquid passing through the membrane, and “retentate” (concentrate) designates the fraction not passing through the membrane.

Reverse osmosis. Reverse osmosis (RO) designates a membrane separation process, driven by a pressure gradient, in which the membrane separates the solvent (generally water) from other components of a solution. With reverse osmosis, the membrane pore size is very small allowing only small amounts of very low molecular weight solutes to pass through the membranes. It is a concentration process using a 100 MW cutoff, 70 psig, temperatures less than 40° C. with cellulose acetate membranes and 70-80° C. with composite membranes. Hyperfiltration is the same as RO.

Ultrafiltration. Ultrafiltration (UF) designates a membrane separation process, driven by a pressure gradient, in which the membrane fractionates components of a liquid as a function of their solvated size and structure. In UF, the membrane pore size is larger allowing some components to pass through the pores with the water. It is a separation/fractionation process using a 10,000 MW cutoff, 40 psig, and temperatures of 50-60° C. with polysulfone membranes. In UF milk, lactose and minerals pass in a 50% separation ratio; for example, in the retentate would be 100% of fat, 100% of protein, 50% of lactose, and 50% of free minerals.

Microfiltration. Microfiltration (MF) designates a membrane separation process similar to UF but with even larger membrane pore size allowing particles in the range of 0.2 to 2 micrometers to pass through. The pressure used is generally lower than that of UF process. MF is used in the dairy industry for making low-heat sterile milk as proteins may pass through but bacteria do not.

Ion Exchange. Fractionation may also be accomplished using ion exchange processing. It relies on inert resins (cellulose or silica based) that can adsorb charged particles at either end of the pH scale. the design can be a batch type, stirred tank or continuous column. The column is more suitable for selective fractionation. Whey protein isolate (WPI), with a greater than 90% protein content, can be produced by this method. Following adsorption and draining of the deproteined whey, the pH or charge properties are altered and proteins are eluted. Protein is recovered from the dilute stream through UF and drying. Selective resins may be used for fractionated protein products or enriched in fraction allow tailoring of ingredients.

Even with use of these techniques typical protein isolates only experience about 80% purity. These techniques also change the protein profile of the resulting isolate so that it no longer mimics that of the original milk product.

For example casein is typically prepared by adjusting the pH of milk to near the isoelectric pH of casein at which pH the casein precipitates and can be collected free of other soluble components of milk including whey proteins (see Schwartz, Encyclopedia of Food Science and Technology, W. H. Hui, Editor, Wiley & Sons, 1991, Vol. 1, pages 310-318). In a variation of the precipitation technology, Connelly (U.S. Pat. No. 4,376,020) discloses that the whey proteins can be made to interact with casein by treating the milk with alkaline and acid pH adjustments. The whey casein complex thus prepared is precipitated near the isoelectric pH of casein and the precipitated complex is collected and washed free of other soluble components. Grafferty and Mulvihill (Journal of Soc. Dairy Technology, 40, 82-85, 1997) reported on the recovery of milk proteins by acid precipitation (pH 4.6) of casein and whey protein complexes formed by heating milk at an alkaline pH. The insoluble acid precipitates were made into functional ingredients for foods by neutralizing with food grade alkaline agents.

Another type of precipitated whey casein complex is described by DeBoer (U.S. Pat. No. 3,882,256) wherein milk is heated to greater than 90° C. to form the whey casein complex. Calcium chlorine is added to the heated milk to precipitate the whey casein complex and the precipitate is washed free of other soluble components from milk. Commercial ingredients made by these precipitation and soluble whey technologies are typically called “caseinate” “total milk protein” “milk protein co-precipitate” or “milk protein isolate”.

Alternatively the whey protein and casein protein can simultaneously be separated from the small molecular whey components of milk (lactose, soluble minerals, peptides, nucleic acids, etc.) using porous membrane filters in a process called ultrafiltration (UF). Milk proteins are concentrated by applying pressure to the milk to force water and low molecular weight components through the porous membrane filter while the proteins, fat and insoluble minerals are retained. Material passing through the membrane is termed the permeate and the material not passing through the membrane is termed the retentate. Typically milk proteins are concentrated by UF to a concentration two to five-fold over the level in starting milk. There is a limit to the potential concentration due to the viscosity of the retentate and the dynamics of the membrane filtration process.

Diafiltration (DF) is a similar membrane filtration process wherein water or other diluent is added to the concentrated retentate at or about the same rate that the permeate is removed. Thus the volume of the retentate may not change much during the process of diafiltration, but the low molecular weight materials are continuously removed from the high molecular weight components in the retentate. In the process described by Buhler, et al. (U.S. Pat. No. 4,125,527) ordinary milk, heated milk, milk plus added whey, and milk adjusted to an acidic pH to enhance the removal of otherwise insoluble calcium salts, were processed by ultrafiltration and diafiltration techniques to concentrate and separate the whey and casein in the retinate using a membrane designed to retain components greater than 10,000 molecular weight. U.S. Pat. No. 6,139,901 describes a process of enhanced separation of small molecular weight and large molecular weight components of milk by adding alkali to adjust the pH above the natural pH of milk and below about pH 10 and heating the composition, thereafter the pH and heated milk is cooled and ultrafiltrated and diafiltrated at near neutral pH or slightly acidic pH. Thus enhancing the separation of small and large molecular weight components and providing a filtration retentate that is a protein concentrate. Furthermore, selection processing conditions, combined with selection of UF and DF membranes, provides one or more permeate compositions with unique protein composition and the permeates can be further processed using current art to provide unique nutritional and functional protein ingredients such as fractionation.

In addition to providing protein concentrates of all proteins from milk and colostrum these materials are often further processed to provide isolation of specific protein components from the starting milk compositions. For example, recent advances in the field of molecular biology allow the production of transgenic animals allowing for the recovery of recombinant protein by the animal. Differences in the regulation of foreign genes in different cell types makes it possible to promote differential expression of a foreign gene in preselected tissue, such as a mammary gland so that a desired recombinant protein, such as BSA, HSA, or lactoferrin, etc. can be produced in the milk of a transgenic animal and then harvested. Transgenic animals provide an advantage for isolation of important proteins in large amounts, particularly by economical purification methods. Such proteins are typically exogenous to the transgenic animal and may comprise pharmaceuticals, food additives, nutritional supplements and the like.

Printed publication WO91/08216 discloses a production of a transgenic bovine species containing a transgene encoding a human lactoferrin polypeptide targeted for expression in mammary secreting cells, the disclosure of which is incorporated herein by reference. Thus the transgenic cattle excretes milk which includes transgenic human lactoferrin. U.S. Pat. No. 5,919,913 also incorporated herein discloses a highly complex process for isolating human lactoferrin from such a transgenic bovine. The rate-limiting step in mass production of these proteins now is the economical isolation and recovery of the proteins free from other contaminants present in milk or even closely related bovine homologies of the recombinant protein.

As can be seen, a need exists in the art for simple processes for isolating proteins from milk components. It is an object of the present invention to provide a process for clarification and purification of protein isolates from milk, whey, colostrum, and the like.

It is yet another object of the invention to provide methods for isolating transgenic proteins from milk produced by animals engineered to express the same.

It is yet another object of the invention to provide methods for isolating proteins from sweet and acid whey for use in pharmaceutical, nutritional and other formulations.

It is yet another object of the invention to provide a fast, economic methodology for recovering protein isolates from milk products.

It is yet another objection of the invention to provide for a protein isolate which closely mirrors the protein profile of the starting material.

It is yet another object of the invention to provide a protein isolate which is 90% or greater pure protein.

It is yet another object of the invention to provide an isolation process which does not damage or denature significant proteins present in the starting material.

### SUMMARY OF THE INVENTION

According to the invention, Applicants have identified a clarification process which is simple and quick and provides a protein isolate of greater than 90% whey protein which may then optionally be further purified to select out individual proteins. The process involves a number of steps, the order of which is critical, and novel lipid removal techniques to achieving the highly pure and clarified protein isolate of the invention.

According to the invention, the milk starting material must first have any cream component, if present, removed. This may be done quickly and simply by centrifuging the milk material and skimming off the cream. It may also be accomplished by letting the milk set, as the milk will naturally separate with the cream rising to the surface. This step is not performed if the starting material is a whey product, as these products already have cream removed. In the method of the invention the de-creamed milk then has the casein removed. Caseins are removed by pH adjustment or any of a number of methods known in the art including filtration, filter pressing or centrifugation after casein precipitation. It is essential for the invention that the casein material be removed prior to the remaining steps. Casein removal occurring after any of the remaining steps was found by the inventors to decrease the purity of the isolate and the purity level was not obtained.

In a next step, a silica material is added to clarify the isolate and remove lipids with centrifugation. The product is clarified at this point. Next, further lipids may be removed by means known in the art such as water dialysis. It is essential that the caseins be removed prior to removal of the lipid (fat) component of the milk, as reversal of these two steps will not result in a protein filtrate of greater than 80% (preferrably 90%) as specified by the invention. The resulting isolate also mirrors the protein of the profile starting material.

Finally the ultimate product may be filtered to isolate a particular protein product. This process may be used for purification of any protein from the starting material and is particularly useful for isolation of transgenic proteins which have been produced and expressed in excreted milk from a transgenic bovine species. The product is also acceptable for food grade preparations achieving a purity and clarity that is required for processing of the same.

Starting material can include milk, colostrum, whey, or any other composition capable of being produced and excreted from the mammary glands of any lactating animal, which includes but is not limited to cows, goats and humans.

Definitions

Unless defined otherwise all technical and scientific terms used herein shall have their same meaning as commonly understood by one of ordinary skill in the art to which this invention pertains. Although any methods and materials similar or equivalent to those described herein can be used in the practice or testing of the present invention, the preferred methods and materials are described. For purposes of the present invention, the following terms are defined below.

The term “naturally-occurring” as used herein as applied to an object refers to the fact that object can be found in nature. For example, a polypeptide or polynucleotide sequence that is present in an organism (including viruses) that can be isolated from a source in nature and which has not been intentionally modified by man in a laboratory, is naturally-occurring.

As used herein, “substantially pure” means that an object species is the predominant species present (i.e. on a molar basis, it is more abundant than any other individual species in the composition), and preferably as substantially purified fraction is a composition wherein the object species comprises at least about 50% (on a molar basis) of all macro molecular species present. Generally a substantially pure composition will comprise more than about 80-90% of all macro molecular species present in the composition. Most preferably the object species is purified to essential homogeneity (contaminant species cannot be detected in the composition by conventional detection methods) wherein the composition consists essentially of a single macro molecular species.

As used herein, the term “enriched” refers to a composition or fraction wherein an object species has been partially purified such that, on a molar ratio basis, at least about 10% of one or more naturally occurring contaminant species has been removed. For example a sample from milk of a transgenic bovine expressing human lactoferrin may be enriched for human lactoferrin by selectively removing caseins by acid precipitation (e.g., the whey fraction is thereby enriched for human lactoferrin).

As used herein, the term “milk” shall include any composition capable of being excreted from the mammary gland of a lactating animal.

As used herein, the term “milk starting material” shall be intended to include milk as defined above including colostrum as well as components thereof including whey such as sweet whey or acid whey, or other partially purified products. Milk starting material may also include normaturally occuring proteins such as recombinant proteins.

As used herein, the term “whey protein isolate” shall be intended to include an isolate having at least about 80% protein content and preferrably 90% or greater protein content. A “whey protein isolate” prepared according to the invention can include recombinant as well as naturally ocurring proteins.

The protein isolate of the invention is substantially purified by application of the process of the invention. That is, the protein isolate is substantially free from contamination with lactose, fat, and ash present in milk. Protein products isolated by the method of the invention are suitable for a formulation and pharmaceutical or nutrient supplements comprising the protein isolate and typically comprising from at least about 1 mg to several grams or more of the protein per dose. For a non limiting example as disclosed herein, recombinant human serum albumin may be recovered by the methods herein which has been produced by transgenic cows. This product has numerous scientific and pharmaceutical uses including drug therapy and blood transfusion in humans which has any of a number of important scientific nutritional and pharmaceutical uses.

Pharmaceutical compositions comprising the protein isolate of the invention can be formulating using standard techniques and typically involve the combination of the isolate with a physiologically compatible carrier for administration.

### BRIEF DESCRIPTION OF THE DRAWINGS

FIG. 1 is a diagram depicting the essential steps of the clarification process.

FIG. 2 is a gel showing the electrophoretic separation of the protein components of whey.

FIG. 3 is a gel showing the proteins present at different stages of the process of the invention.

A “plus sign (+)” denotes sample was run reduced. Refers to sample treatment where non-reduced means the sample was treated with SDS before applying to the gel and reduced means the sample was treated with both SDS and 2-mercaptoethanol (a commonly employed reducing agent that reduces disulfide bonds).

Lane 2 and 3 is the starting material for protein isolate of the invention shown in lanes 4 & 5. Protein composition is essentially unchanged. “High molecular weight aggregate peak” at the top of gel in lane 2 is removed. Overall less background staining is observed and bands appear somewhat crisper when residual lipids and denatured proteins are removed.

Lanes 6 and 7 is a commercially available ion-exchanged whey protein isolate. It appears to have a reduced IgG concentration and an enriched beta-lactoglobulin and alpha-lactalbumin content.

Lanes 8 and 9 is a commercially available microfiltered whey protein isolate. It appears to have a reduced IgG concentration as well as reduced albumin level. This results in a higher percentage of beta-lactoglobulin and alpha-lactalbumin present.

| | | TP | SALINE | SDS BUF. |
|---|---|---|---|---|
| WELL | SAMPLE | mg/ml | DIL. | DIL. |
| 1 | MW Marker | n/a | n/a | 1:20 |
| 2 | Current Product WPC | 80 | 1:4 | 1:4 |
| 3 | + | 80 | 1:4 | 1:4 |
| 4 | Our New WPI | 92 | 1:4.6 | 1:4 |
| 5 | + | 92 | 1:4.6 | 1:4 |
| 6 | Ion-Exchanged WPI | 86 | 1:4.3 | 1:4 |
| 7 | + | 86 | 1:4.3 | 1:4 |
| 8 | Microfiltered WPI | 86 | 1:4.3 | 1:4 |
| 9 | + | 86 | 1:4.3 | 1:4 |
| 10 | MW Marker | n/a | n/a | 1:20 |

### DETAILED DESCRIPTION OF THE INVENTION

According to the invention a novel process is disclosed for the preparation of a protein isolate from milk starting material. As explained earlier this starting material can be naturally occurring transgenic or even processed milk. The starting material can include but is not limited to: normal milk from mammals (cows, goats, humans), colostrum from mammals (cows, goats, humans), transgenic milk from mammals, sweet whey, and acid whey.

Further in a preferred embodiment a novel clarification process for the above feedstreams is disclosed whereby a clear product can be made. Certain modifications are used to apply this process to all of the feedstreams so that the end products can be used for various applications and/or further downstream processing. The resulting product is enriched for protein content and typically is about 80% or greater and preferrably 90% or greater protein content. The protein profile of this isolate closely mirrors that of the altering material making it valuable as milk replacer or supplements. This product may also be further fractionated to isolate a specific protein.

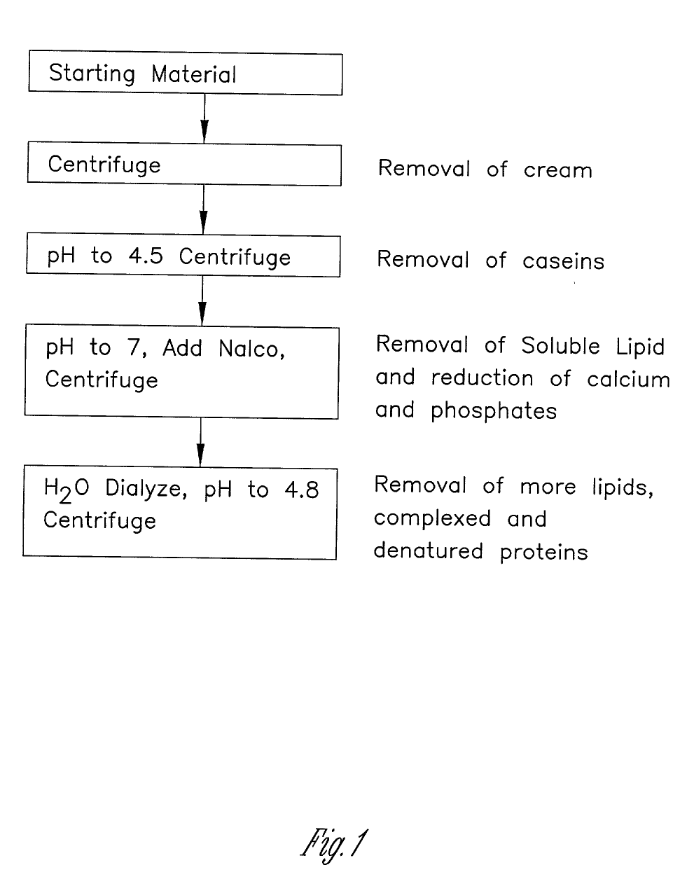

The main process involves the following steps diagrammed in FIG. 1.

First if the starting material is milk, colostrum or other cream containing product, the material must have the cream removed. This can be done by any method known in the art including even simply letting the milk stand undisturbed. While the cream is capable of rising to the top upon standing undisturbed, the cream can be made to rise faster upon centrifugation such that it occurs in a short time that is favorable to processing the milk in a commercial setting. For centrifugation the milk may be centrifuged in a dead-end centrifuge at 7,280 X G-force on the small scale or a cream separator on the large scale. On the small scale, the cream rises to the top and may be skimmed off manually while on the large scale the cream separator is designed to separate mechanically the cream from the milk. The resulting milk separated from the cream is called decreamed milk.

If the milk starting material is colostrum then the colostrum is preferably diluted with water (likely equal parts of water (vol./vol. or wt/wt)) to lower the viscosity since colostrum can be very thick before the cream separation milk starting materials such as sweet whey or acid whey do not have cream in the starting material and do not require this step.

Next the casein is removed from the decreamed milk starting material. Typically this is done by pH adjustment to cause the casein to precipitate. Accordingly the decreamed milk is pH adjusted to approximately 4.40 to 4.60 with an appropriate acid known to those of skill in the art, such as dilute HCl, preferably 10% acetic acid is used. The precipitated caseins can then be removed by either filtration, settling, filter pressing or preferably by centrifugation@7,280 X G-force. After removal of caseins from the milk by this step, the resulting product is referred to as acid whey.

Again if the starting material is sweet whey or acid whey then this step may be skipped since there is virtually no casein, (except that which may be present as residual casein from the cheese process), in the starting material.

The next step involves removal of lipids and optionally the clarification of the product if desired. The acid whey may be pH adjusted and treated with a silica product. Preferred is colloidal silica such as the commercially available product Nalco (Nalco Company, Chicago, Ill.). The acid whey is pH adjusted to between 6.00 and 8.00 and preferably between 6.8 and 7.2 to then colloidal silica, Nalco 1115, is added to the whey at a level sufficient to bind calcium and phosphate from the whey. Typically this will be at a level of approximately 1% (vol/vol) or 1.096% (wt/wt). The solution is mixed and allowed to react for a period of not less than approximately 30 minutes to overnight and after which time the mixture is centrifuged@7,280 X g-force on the small scale or on an industrial centrifuge at appropriate setting to those skilled in the art to separate a clear supernate product. Colloidal silica is a product made from Silicon Dioxide (SiO₂) and any silica product may be used for the invention. Fumed silica may also be used for this application. The silica also removes a portion of calcium and phosphate from the whey feedstream as well as lipids. After the centrifugation, the feedstream is clear. Silica products which may be used include any silicon dioxide product.

The solution is now dialyzed to precipitate any remaining lipids from the mixture. The whey is water dialyzed until a point that conductivity of the solution reaches a low conductivity of less than 4 mS/cm and preferably <1 mS/cm and most preferably 0.6 mS/cm. The solution is now pH adjusted to between 4.6 and 4.8 with an acid and preferably 2M HC 1. Lipids are destabilized when the conductivity is reduced to this point and the pH is adjusted to between 4.6 and 4.8 and they will precipitate. The resulting precipitate can be removed by methods such as filtration, filter pressing, settling or preferably by centrifugation as described earlier. This step also removes any complexed proteins and denatured proteins.

The final product can easily be filtered, typically at 0.2 μm filter, to remove all remaining small molecules. The removal of all of these above mentioned substances results in a crystal clear product with no fouling substances (lipids) to clog a filter. This protein isolate may then be further fractionated or used as is. In one embodiment the whey protein isolate may be spray dried for ease of storage.

One particularly useful application of this process is for purifying recombinant proteins from the milk starting material. The recombinant protein which is usually human needs to be separated from the other cows milk protein and this is a laborious process. The milk must also be clarified so that further purification can take place. This clarification process results in a highly clarified end product which is suitable for column chromatography and other complex downstream processing. When the clarified whey is intended to be taken on to such other purification schemes, the benefits of having such a unique clarified feed stream result in enormous efficiencies in the downstream processing such that the results are so positive and economical that it could not be made possible unless this process was performed first.

The end product may also be a whey protein isolate such as a food ingredient.

### EXAMPLE

The process of the invention was employed as described herein and compared against other commercially available milk starting material protein isolates.

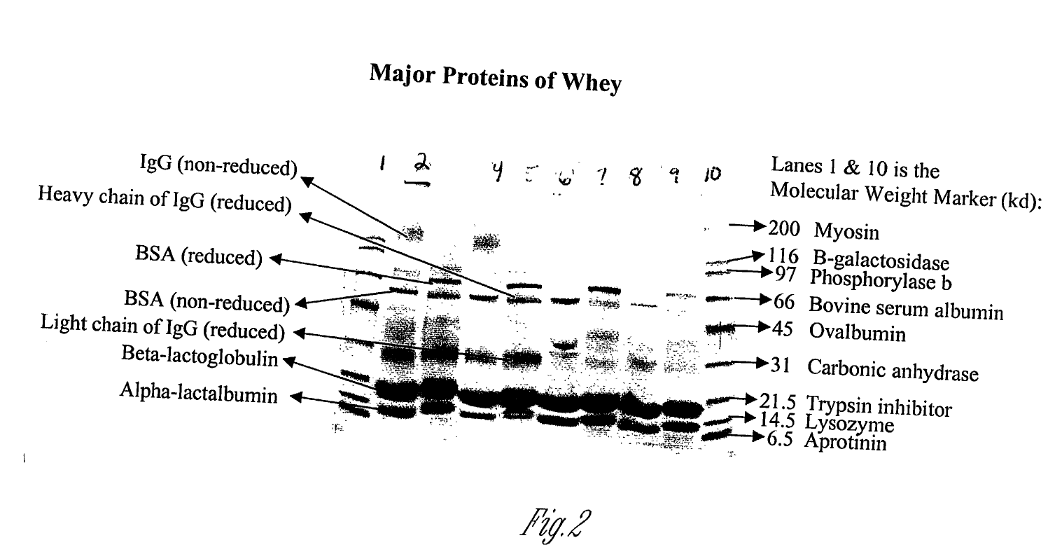

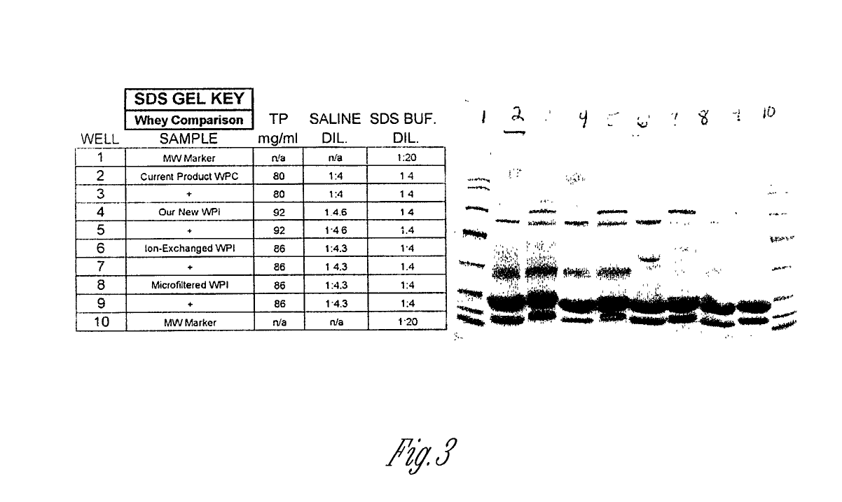

The isolates were subjected to electrophoresis and separated by molecular weight. Sodium dodecyl sulfate-polyacrylamide gel electrophoresis (SDS-PAGE) was run under non-reduced and reduced conditions. Ready gels (4-20% Cross-linking), Mini-PROTEAN II apparatus, and molecular weight marker (Bio-Rad) were used according to the manufacturer's directions. All samples were diluted to 5 mg/ml and 5 μl was applied to each lane. The gel was stained with Gelcode® Coomassie Blue Stain (Pierce) according to manufacturer's directions. FIG. 2 shows a label of the particular protein component of whey associated with a particular band. FIG. 3 is a gel showing the proteins present at different stages of the process of the invention.

A “plus sign (+)” denotes sample was run reduced. Refers to sample treatment where non-reduced means the sample was treated with SDS before applying to the gel and reduced means the sample was treated with both SDS and 2-mercaptoethanol (a commonly employed reducing agent that reduces disulfide bonds).

Lane 2 and 3 is the milk starting material for protein isolate of the invention which is shown in lanes 4 & 5. Protein composition is essentially unchanged. “High molecular weight aggregate peak” at the top of gel in lane 2 is removed. Overall less background staining is observed and bands appear somewhat crisper when residual lipids and denatured proteins are removed.

Lanes 6 and 7 is a commercially available ion-exchanged whey protein isolate. It appears to have a reduced IgG concentration and an enriched beta-lactoglobulin and alpha-lactalbumin content.

Lanes 8 and 9 is a commercially available microfiltered whey protein isolate. It appears to have a reduced IgG concentration as well as reduced albumin level. This results in a higher percentage of beta-lactoglobulin and alpha-lactalbumin present.

| | | TP | SALINE | SDS BUF. |
|---|---|---|---|---|
| WELL | SAMPLE | mg/ml | DIL. | DIL. |
| 1 | MW Marker | n/a | n/a | 1:20 |
| 2 | Current Product WPC | 80 | 1:4 | 1:4 |
| 3 | + | 80 | 1:4 | 1:4 |
| 4 | Our New WPI | 92 | 1:4.6 | 1:4 |
| 5 | + | 92 | 1:4.6 | 1:4 |
| 6 | Ion-Exchanged WPI | 86 | 1:4.3 | 1:4 |
| 7 | + | 86 | 1:4.3 | 1:4 |
| 8 | Microfiltered WPI | 86 | 1:4.3 | 1:4 |
| 9 | + | 86 | 1:4.3 | 1:4 |
| 10 | MW Marker | n/a | n/a | 1:20 |

As can be seen, the whey isolate prepared according to the invention more closely mimics the protein ratios present in the original starting material and has fewer contaminants.

As can be seen from the foregoing, the invention accomplishes at least all of its objectives.

What is claimed is:

1. A method for producing a whey protein isolate from a casein containing milk starting material comprising:
obtaining de-creamed milk starting material;
removing any casein present in said starting material, to form decaseinated milk starting material, and thereafter
removing lipids from said decaseinated starting milk product, to form a protein isolate of 80% or greater protein content.

2. The method of claim 1 wherein said step of removing casein comprises:
adjusting the pH of said milk starting material so that casein will precipitate.

3. The method of claim 2 wherein said pH is adjusted to a pH of approximately 4.4 to 4.6.

4. The method of claim 3 wherein said pH adjustment is through addition of an acid such as dilute HCl and 10% acetic acid.

5. The method of claim 2 wherein said casein removal further comprises the step of removing precipitated caseins.

6. The method of claim 5 wherein said precipitated caseins are removed by a method identified from the group consisting of filtration, settling, filter pressing or centrifugation.

7. The method of claim 6 wherein said precipitated casein is removed by centrifugation.

8. The method of claim 1 wherein said removal of lipids is accomplished by pH adjustment and water dialyzing said solution to precipitate remaining lipids.

9. The method of claim 8 wherein said pH adjustment and water dialysis occurs until a point that the activity of the solution reaches a low conductivity of less than 4 mS.

10. The method of claim 9 wherein said conductivity is in a range of less than 1 mS then the pH is adjusted to between 4.6 and 4.8 with acid, preferably 2M HCl.

11. The method of claim 10 wherein said conductivity of said solution is 0.6 mS.

12. The method of claim 8 further comprising the step of removing said precipitated lipids.

13. The method of claim 12 wherein said precipitated lipids are removed by a process selected from the group consisting of filtration, filter pressing, settling, or centrifugation.

14. The method of claim 1 further comprising the step of:
filtering said protein isolate to remove any remaining small molecules.

15. A method for producing a whey protein isolate from whey starting material comprising:
removing lipids from said whey starting material, by the addition of a silicon dioxide to precipitate said lipids and to clarify said isolate.

16. The method of claim 15 wherein said removal of lipids is accomplished further by the step of:
pH adjusting and water dialyzing said solution to precipitate remaining lipids.

17.
The method of claim 16 wherein said pH adjustment and water dialyzation occurs until a point that the activity of the solution reaches a low conductivity of less than 4 mS.

18. The method of claim 17 wherein said conductivity is in a range of less than 1 mS then the pH is adjusted to between 4.6 and 4.8 with acid, preferably 2M HCl.

19. The method of claim 18 wherein said conductivity of said solution is 0.6 mS.

20. The method of claim 19 further comprising the step of removing said precipitated lipids.

21. The method of claim 17 wherein said precipitated lipids are removed by a process selected from the group consisting of filtration, filter pressing, settling, or centrifugation.

22. The method of claim 15 further comprising the step of filtering said clarified isolate to remove any remaining small molecules.

23. A method for producing a clarified whey protein isolate from a milk starting material comprising:
obtaining de-creamed milk starting material;
removing any casein present in said starting material, to form decaseinated milk material and thereafter
introducing an effective amount of silica to said decaseinated starting material so that lipids, calcium and phosphate may be precipitated and removed, to form a clarified product, and
further removing lipids from said clarified product.

24. The method of claim 23 wherein said step of removing casein comprises adjusting the pH of said milk starting material so that casein will precipitate.

25. The method of claim 24 wherein said pH is adjusted to a pH of approximately 4.4 to 4.6.

26. The method of claim 25 wherein said pH adjustment is through addition of an acid such as dilute HCl and 10% acetic acid.

27. The method of claim 24 wherein said casein removal further comprises the step of removing precipitated caseins.

28. The method of claim 27 wherein said precipitated caseins are removed by a method identified from the group consisting of filtration, settling, filter pressing or centrifugation.

29. The method of claim 28 wherein said precipitated casein is removed by centrifugation.

30. The method of claim 23 wherein said further removal of lipids is accomplished by water dialyzing and subsequent pH adjustment of said solution to precipitate lipids.

31. The method of claim 30 wherein water dialyzation occurs until a point that the activity of the solution reaches a low conductivity of less than 4 mS.

32. The method of claim 31 wherein said conductivity is in a range of less than 1 mS then the pH is adjusted to between 4.6 and 4.8 with acid, preferably 2M HCl.

33. The method of claim 32 wherein said conductivity of said solution is 0.6 mS.

34. The method of claim 30 further comprising the step of removing said precipitated lipids.

35. The method of claim 34 wherein said precipitated lipids are removed by a process selected from the group consisting of filtration, filter pressing, settling, or centrifugation.

36. The method of claim 23 further comprising the step of filtering said clarified product to remove any remaining small molecules.

37. A whey protein isolate product purified from milk starting material produced by the method of claim 1.

38. A whey protein isolate prepared from milk starting material, said isolate produced by the method of claim 15.

39. A whey protein isolate prepared from milk starting material, said isolate produced by the method of claim 23.

40. A whey protein isolate produced from milk starting material, wherein said isolate comprises approximately 80% or greater and preferably 90% or greater protein content of said milk starting material, and wherein said protein isolate mimics the protein profile present in said starting milk material.

41. A method for purifying a protein from casein containing milk starting material comprising:
obtaining de-creamed milk starting material;
removing any casein present in said starting material, to form decaseinated milk starting material, and thereafter
removing lipids from said decaseinated starting milk product, to form a protein isolate of 80% or greater and preferably 90% or greater protein content, and therafter
purifying said protein from the protein isolate.

42. A method for purifying a recombinant protein from casein containing milk starting material comprising:
obtaining de-creamed milk starting material;
removing any casein present in said starting material, to form decaseinated milk starting material, and thereafter
removing lipids from said decaseinated starting milk product, to form a protein isolate of 80% or greater and preferably 90% or greater protein content, and therafter
purifying said recombinant protein from the protein isolate.

43. The method of claim 43 wherein said recombinant protein is recombinant human serum albumin.
